(19) United States
(12) Patent Application Publication
(10) Pub. No.: US 2001/0027297 A1
(43) Pub. Date: Oct. 4, 2001

(54) CATHETER

(75) Inventor: MASAKI ITO, AICHI-KEN (JP)

Correspondence Address: SUGHRUE MION ZINN MACPEAK & SEAS, 2100 PENNSYLVANIA AVENUE N W, WASHINGTON, DC, 200373213

(73) Assignee: Robert V. Sloan

(21) Appl. No.: 09/223,391

(22) Filed: Dec. 30, 1998

(51) Int. Cl. A61M 5/178

(52) U.S. Cl. 604/165.01

(57) ABSTRACT

A catheter has an outer tube capable of turning an inner tube smoothly. The catheter comprises an inner tube 11 having a leading end portion formed into a suitable shape matching an affected part 18, an outer tube 12 fitted movably on the inner tube 11 and a joint mechanism for enabling the inner tube 11 to move integrally with the outer tube 12 without preventing the movement of the outer tube 12.

### BACKGROUND OF THE INVENTION

1. Field of the Invention

The present invention relates to a catheter and, more particularly, to a catheter capable of changing the direction of the leading end portion of an inner tube, guided in an outer tube, in a human body.

2. Description of Related Art

The catheter of the aforementioned type is disclosed in Unexamined Published Japanese Patent Application No. 2-13472 or 4-231068, for example. In this catheter of the prior art, the leading end portion of the inner tube can be changed in direction by turning the inner tube relative to the outer tube which is held stationary. Moreover, a clearance is left between the inner tube and the outer tube so as to allow turning of the former relative to the latter.

However, the aforementioned catheter must have an external diameter as small as possible in its entirety and the internal diameter of the inner tube must have an internal diameter as large as possible due to the necessity of maintaining a desired flow rate of a contrast medium through the inner tube. The clearance is made as small as possible in the manufacture because of these conflicting demands. When the catheter is brought into actual use even if the inner tube and the outer tube are made of materials having low coefficients of friction, a drawback arises since the inner tube interferes with the stationary outer tube so that the turning motion of the inner tube relative to the outer tube cannot be effected smoothly. Since the relative position of the outer tube to the inner tube is not fixed, they may go out of the desired position relative to each other while the catheter is being used. Since the relative position of the outer tube to the inner tube is not indicated, the catheter is not convenient to use.

### SUMMARY OF THE INVENTION

Therefore, the object of the present invention is to provide a catheter having none of the foregoing defects.

In order to achieve this object, according to a first aspect of the invention, there is provided a catheter comprising: an inner tube having a leading end portion formed into a suitable shape matching an affected part; an outer tube fitted movably on said inner tube; and a joint mechanism for enabling said inner tube to move integrally with said outer tube without preventing the movement of said outer tube. Thus, the change in the direction of the inner tube is made by the integral turning motion with the outer tube so that it can be made smoother than the case when the inner tube turns relative to the outer tube.

According to a second aspect of the invention, there is provided a catheter comprising: an inner tube having a leading end portion formed into a suitable shape matching an affected part; an outer tube fitted movably on said inner tube; and a lock mechanism for stopping/fixing the movement of said outer tube at an arbitrary position. Thus, the movement of the outer tube on the inner tube can be stopped/fixed at an arbitrary position by the lock mechanism so that the catheter can be prevented from going out of position while it is used.

According to a third aspect of the invention, there is provided a catheter comprising: an inner tube having a leading end portion formed into a suitable shape matching an affected part; an outer tube fitted movably on said inner tube; and an indication mechanism for indicating the stroke of the movement of said outer tube. Thus, the stroke movement of the outer tube on the inner tube can be indicated by the indication mechanism so that the catheter can be easily handled.

### BRIEF DESCRIPTION OF THE DRAWINGS

FIG. 1 is a side elevation showing a catheter according to a first embodiment of the invention;

FIG. 2 is a section taken along line A-A of FIG. 1;

FIG. 3 is a view showing the state in which the catheter shown in FIG. 1 is used;

FIG. 4 is a side elevation showing a catheter according to a second embodiment of the invention;

FIG. 5 is a section taken along line B-B of FIG. 4;

FIG. 6 is a section taken along line C-C of FIG. 4;

FIG. 7 is a side elevation showing a catheter according to a third embodiment of the invention;

FIG. 8 is a section taken along line D-D of FIG. 7;

FIG. 9 is a section showing a modification of the structure shown in FIG. 8;

FIG. 10 is a side elevation showing a catheter according to a fourth embodiment of the invention;

FIG. 11 is a side elevation showing a catheter according to a fifth embodiment of the invention;

FIG. 12 is a section taken along line E-E of FIG. 11;

FIG. 13 is a section showing a first embodiment of a lock mechanism;

FIG. 14 is a section showing the lock mechanism of FIG. 13 in a locking state;

FIG. 15 is a perspective view showing a tube to be used in the lock mechanism shown in FIG. 13;

FIG. 16 is a perspective view showing a second mode of a lock mechanism;

FIG. 17 is a side elevation showing a third embodiment of a lock mechanism;

FIG. 18 is a section of the lock mechanism shown in FIG. 17;

FIG. 19 is a side elevation showing a fourth embodiment of a lock mechanism;

FIG. 20 is a section showing a fifth embodiment of a lock mechanism;

FIG. 21 is a section showing a sixth embodiment of a lock mechanism; and

FIG. 22 is a section showing a seventh embodiment of a lock mechanism.

### DESCRIPTION OF THE PREFERRED EMBODIMENTS

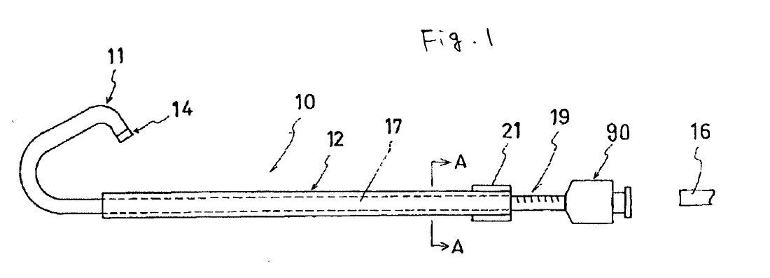

In FIGS. 1 and 2, a catheter 10 is provided with an inner tube 11. The leading end portion, as located at the lefthand end portion of FIG. 1 of the inner tube 11, is suitably bent into a desired shape according to the related part (e.g., a blood vessel part to be inspected or treated) of a patient. On this inner tube 11, there is fitted an outer tube 12 which is formed in a cylindrical shape so as to move freely in the axial directions. As will be described hereinafter, the inner tube 11 can turn integrally with the outer tube 12. This outer tube 12 is equipped at its base end portion, as located at the righthand end portion of FIG. 1, with a grip member 21 to allow easy gripping of the catheter 10.

As clearly seen, especially from FIG. 2, the outer tube 12 has a round section, but a guide hole 13 extending axially through the outer tube 12 has a rectangular or square section. Thus, the inner tube 11 has a rectangular or square section dimensioned to leave a clearance 15 between the inner periphery of the guide hole 13 of the outer tube 12 and the outer periphery of the inner tube 11 which allows the inner tube to move axially through the guide hole 13. By thus shaping the section of the inner tube 11 and the guide hole 13 of the outer tube 12, the inner tube 11 and the outer tube 12 are turned together when either of them is turned. Therefore, it is necessary to maintain the accuracy of the clearance 15 to warrant a smooth movement of the inner tube 11 along the guide hole 13. This accuracy of clearance is far better than when the inner tube 11 is enabled to turn without any friction within the guide hole 13.

The base end portion, as located on the righthand end portion of FIG. 1, of the inner tube 11 is adapted to be connected through a connector 90 to another tube 16. This tube 16 is jointed to a syringe (not shown) so that a contrast medium, for example, is fed into an inner passage 17 extending axially in the inner tube 11. As shown in FIG. 3, the catheter 10 is inserted into the body by a well-known method, and the inner tube 11 is then moved axially and turned so that the leading end portion of the inner tube 11 can be brought to a position near the part 18 to be inspected. At this position, the contrast medium, as having passed through the inner passage 17, is fed from the leading end portion of the inner tube 11 to the part 18.

On the base end portion of the inner tube 11, there is engraved or indicated a scale 19 which allows an easy determination as to how far the outer tube 12 has moved on the inner tube 11. At the leading end portion of the inner tube 11, on the other hand, there is mounted a soft tip 14. This soft tip 14 softens as much as possible any impact when the leading end portion of the inner tube 11 comes into abutment against a blood vessel wall during the use of the catheter 10. If the external diameter of the soft tip 14 is suitably adjusted, the outer tube 12 comes, when moved leftward, into engagement at its leading end portion (or its lefthand end portion) with the leading end portion of the inner tube 11 so that its further movement in the leftward direction is prevented. As a result, the movement of the outer tube 12 is confined between the connector 90 and the soft tip 14 so that the outer tube 12 is prevented from coming off the inner tube 11.

The inner tube 11 and the outer tube 12 may be identical in their materials and internal structures to those of the prior art. Specifically, the material can be exemplified by polyamide, polyurethane or polyethylene, or a mixture of the recited polymers as the case may be, such as when a higher bending strength or hardness is demanded. On the other hand, the internal structure may be exemplified by a single-layered tube or a two- or more-layered tube. In the latter case, a mesh of stainless steel or another metal may be sandwiched between the two adjoining layers. In order to observe the catheter clearly in an X-ray examination, the inner tube 11 (or the outer tube 12, as the case may be) may contain a material such as an X-ray impermeable substance such as barium.

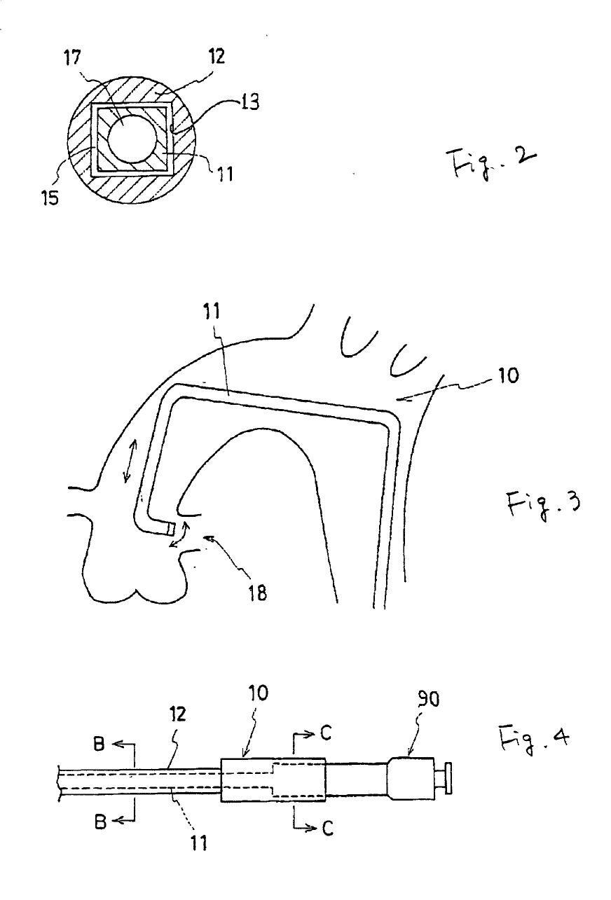

In the catheter 10 thus far described, both the sectional shape of the guide hole 13 extending axially through the outer tube 12 and the sectional shape of the inner tube 11 are rectangular or square throughout the entire length to facilitate axial movement along the guide hole 13. This structure is adopted because both tubes are turned simultaneously or integrally when either of them is turned. The invention should not be limited to this structure, but may adopt another structure as long as the simultaneous or integral turns of the tubes are ensured. As shown in FIGS. 4 to 6, both the sectional shape of the guide hole 13 extending axially through the outer tube 12 and the sectional shape of the inner tube 11, to facilitate axial movement along the guide hole 13, are made partially rectangular or square. In the remaining portions, however, both the sectional shape of the guide hole 13 extending axially through the outer tube 12 and the sectional shape of the inner tube 11 may be made circular to move axially along the guide hole 13. In this case, the sectional shape of the lefthand side of the outer tube 12 is also circular.

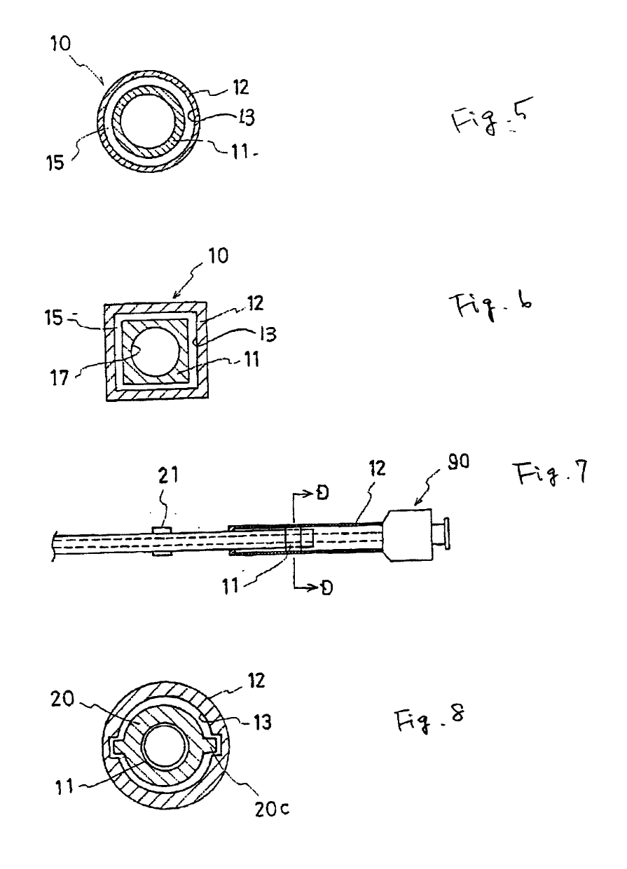

On the outer circumference of the inner tube 11 having a round sectional shape, as shown in FIGS. 7 and 8, there is fixed a member 20 having keys 20a or an oval member 30 as shown in FIG. 9. The sectional shape of the guide hole 13 extending axially through the outer tube 12 is formed into a shape similar to that of the member 20 or the member 30 so that the inner tube 11 and the outer tube 12 can be turned simultaneously or integrally. In the device shown in FIG. 7, a grip member 21 is fixed on the inner tube 11.

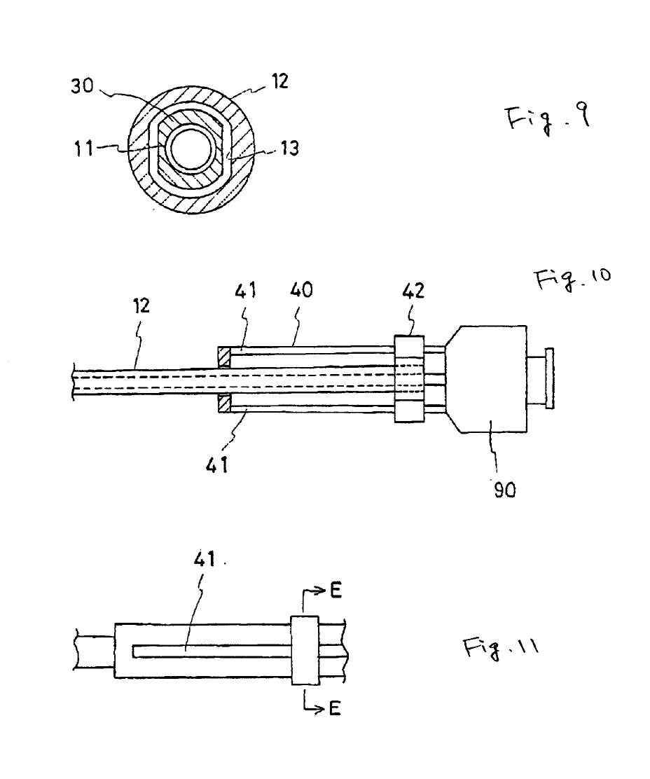

In a bracket 40 arranged outside of the outer tube 12, as shown in FIGS. 10 to 12, there are formed a pair of slits 41 extending axially. A member 42 is comprised of two concentric rings connected by two radial ribs which are guided in the slits 41. The inner ring is connected to the outer side of the outer tube 12. Moreover, the bracket 40 is integrally connected at its righthand end portion with the connector 90. By adopting this structure, by operating only the member 42, the outer tube 12 and the inner tube 11 can be turned integrally and the outer tube 12 can be moved axially. The shape of the slit 40 should not be limited to the shown straight one but may be helical or stepped.

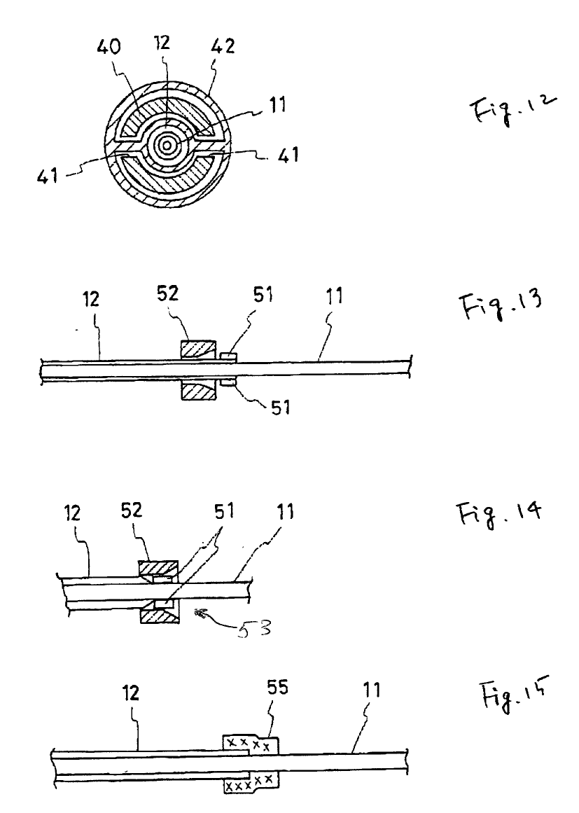

On the outer tube 12, as shown in FIG. 13, there are integrally formed a pair of projections 51. On the outer tube 12, there is also a ring member 52 which is made axially movable relative to the tube 12. When the ring member 52 moves axially toward the projections 51 and rides on them, as shown in FIG. 14, the outer tube 12 is radially reduced to clamp onto the inner tube 11. Thus, the inner tube 11 is locked by a lock mechanism 53 which is composed of the ring member 52 and the paired projections 51, so that the outer tube 12 can be fixed at an arbitrary position.

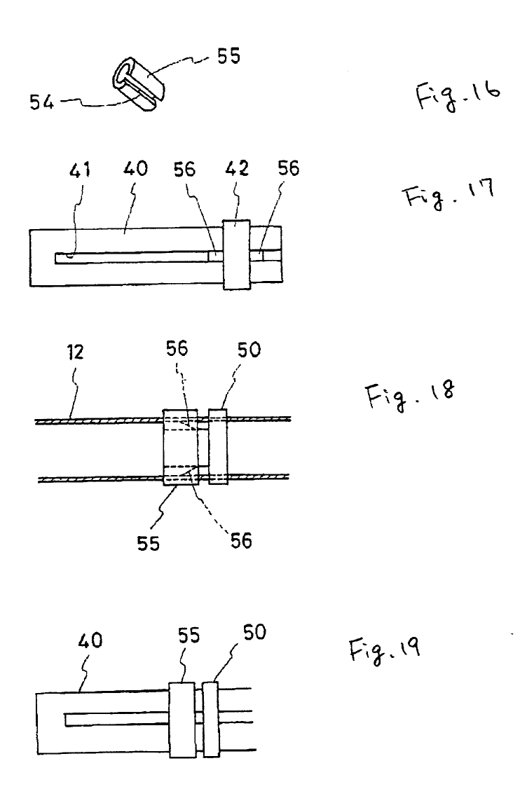

The lock mechanism for fixing the outer tube 12 at an arbitrary position relative to the inner tube 11 may be exemplified by a tube 55 which is made of an elastic material and has an expansion slot 54, as shown in FIGS. 15 and 16.

A second example of the lock mechanism 53 is shown in FIG. 17. In the structure shown in FIGS. 10 to 12, a pair of wedges 56, formed on the opposite sides of the member 42, are fitted in the slits 41.

A third example of the lock mechanism 53 is shown in FIGS. 18 and 19. In the structure shown in FIGS. 10 to 12, the wedges 56, as formed integrally with a moving member 50, are fitted axially in the inner circumference of the member 55 which is fixed on the bracket 40.

A fourth example of the lock mechanism 53 is shown in FIG. 20. In the structure shown in FIGS. 10 to 12, a cylindrical portion 43 having an expansion slot 42a is made integrally with the member 42. A nut member 60 fitted on the cylindrical portion 43 is fastened on a threaded portion 61 of the cylindrical portion 43. The cylindrical portion is radially reduced, as shown in FIG. 21 as a result of a camming action by the nut member 60, to grip the tube 12 to prevent the member 42 from moving on the bracket 40. As a result, the outer inner tube 12 is locked.

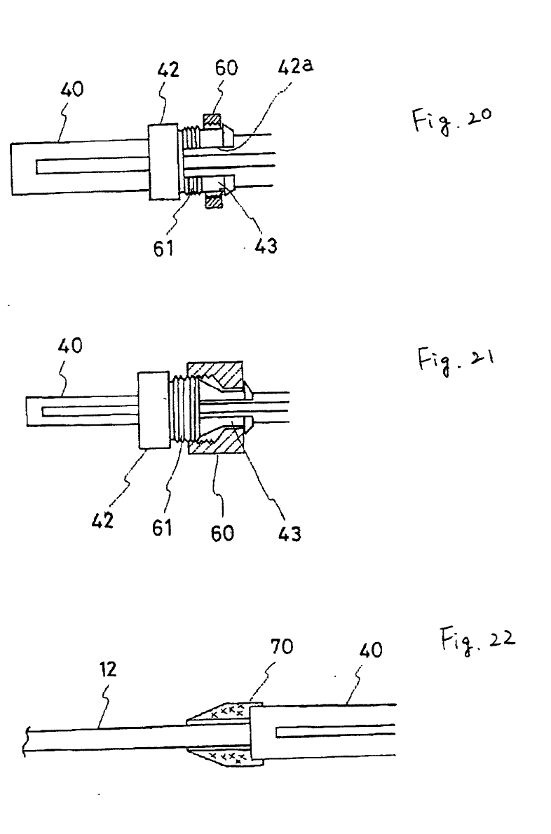

The bracket 40 shown in FIGS. 10 to 12, is shown in FIG. 22 and is provided with an elastic joint 70 which is interposed between the bracket 40 and the outer tube 12 to prevent the catheter 10 from being damaged when bent.

While the preferred embodiments have been described, variations thereto will occur to those skilled in the art within the scope of the present inventive concepts which are delineated by the following claims.

What is claimed is:

1. A catheter comprising an inner tube having a leading end portion formed into a suitable shape matching an affected part, an outer tube fitted movably on said inner tube and a joint mechanism for enabling said inner tube to move integrally with said outer tube without preventing longitudinal movement of said outer tube relative to the inner tube.

2. A catheter comprising an inner tube having a leading end portion formed into a suitable shape matching an affected part, an outer tube fitted movably on said inner tube and a lock mechanism for fixing said outer tube to said inner tube at an arbitrary position.

3. A catheter comprising an inner tube having a leading end portion formed into a suitable shape matching an affected part, an outer tube fitted movably on said inner tube and an indicator mechanism for indicating the stroke of the movement of said outer tube.
